(19) United States
(12) Patent Application Publication
(10) Pub. No.: US 2006/0173180 A1
(43) Pub. Date: Aug. 3, 2006

(54) Aminoalkoxyphenyl indolone derivatives

(75) Inventors: Michael Konkel, Fair Lawn, NJ (US); Mathivanan Packiarajan, Saddle Brook, NJ (US); Heidi Chen, Fort Lee, NJ (US); Upendra Topiwala, North Haledon, NJ (US); Hermogenes Jimenez, Belleville, NJ (US); Jamie Talisman, New York, NY (US)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. Lundbeck A/S

(21) Appl. No.: 11/343,810

(22) Filed: Jan. 31, 2006

Related U.S. Application Data
(60) Provisional application No. 60/649,200, filed on Feb. 2, 2005.

(51) Int. Cl. C07D 413/02; C07D 403/02

(52) U.S. Cl. 544/144; 544/373; 548/465; 548/482

(57) ABSTRACT

This invention is directed to aminoalkoxyphenyl indolone derivatives, which are ligands at the GAL₃ receptor. The invention provides a pharmaceutical composition comprising a therapeutically effective amount of a compound of the invention and a pharmaceutically acceptable carrier. This invention also provides a pharmaceutical composition made by admixing a therapeutically effective amount of a compound of the invention and a pharmaceutically acceptable carrier. This invention further provides a process for making a pharmaceutical composition comprising combining a therapeutically effective amount of a compound of the invention and a pharmaceutically acceptable carrier. This invention also provides a method of treating a subject suffering from depression and/or anxiety which comprises administering to the subject an amount of a compound of the subject invention.

This application claims the benefit of U.S. Provisional Application No. 60/649200, filed Feb. 2, 2005, the contents of which are hereby incorporated by reference.

### FIELD OF THE INVENTION

The present invention relates to compounds that are ligands at the GAL₃ receptor, and as such are useful to treat depression or anxiety.

### BACKGROUND OF THE INVENTION

Throughout this application, various publications are referenced to in full citations. The disclosures of these publications are hereby incorporated by reference into this application to describe more fully the state of the art to which this invention pertains.

Galanin is a 29-30 amino acid neuropeptide that is expressed by neurons in the brain, spinal cord and ganglia of the peripheral autonomic nervous system. Mammalian galanin is conserved between human, rat and mouse, exhibiting almost 90% amino acid homology among species, and the effects of galanin are mediated through receptors that belong in the superfamily of G protein-coupled receptors. Presently, three human galanin receptor subtypes have been cloned and characterized: GALR1 (E. Habert-Ortoli, et al., Proc. Natl. Acad. Sci., 1994, 9, 9780-9783); GALR2 (B. Borowsky, et al., Peptides, 2003, 19, 1771-1781); and GALR3 (K. E. Smith, et al., J. Biol. Chem., 1998, 273, 23321-23326).

The compounds of the present invention are ligands at the human galanin receptor subtype named “human GAL₃ receptor”. The human GAL₃ receptor, whose official gene symbol is GALR3 (see U.S. Pat. No. 6,329,197), has not been assigned an official International Union of Pharmacology (IUPHAR) nomenclature. For the purpose of clarity, the IUPHAR “provisional” name for the human GAL₃ receptor will be used throughout this application.

Data from pre-clinical behavioral studies, in addition to articles in the literature, evidence that targeting the galanin system is of therapeutic benefit in treating depressive and anxiety disorders. Researchers have suggested that blocking the inhibitory effects of galanin on monoamine neurotransmission with galanin receptor antagonists would be predicted to mimic or augment the action of antidepressants. In this context, central administration of galanin was found to attenuate antidepressant-induced increases in rat forebrain levels of 5-HT and noradrenaline (T. Yoshitake, et al., Neurosci. Lett., 2003, 339, 239-242).

Furthermore, it was observed that exogenous galanin alters anxiety-like behavior in rats. Research groups also observed that exogenous galanin activity in the amygdala is associated with anxiogenic-like effects under conditions of stress and high noradrenergic activity (D. A. Morilak, et al., Life Sci., 2003, 73, 715-726).

The link between the GAL₃ receptor and the effects of galanin on depression and anxiety is further evidenced from the evaluation of effects produced by selective GAL₃ small molecule ligands in behavioral models of depression or anxiety: the rat forced-swim and rat social interaction test, respectively. Administration of GAL₃ selective small molecule ligands produces a profile similar to clinically used antidepressants and anxiolytics in behavioral models of depression and anxiety (T. Blackburn, et al., PCT International Application No. PCT/US02/04608). These observations evidence that selective GAL₃ small molecule ligands are useful to treat depression and anxiety.

Current treatments for depression and anxiety are on the market. However, numerous patients do not respond to current treatments. Hence, there remains the need for alternative methods of treatment.

### SUMMARY OF THE INVENTION

The objective of the present invention is to provide compounds that are ligands at the GAL₃ receptor. The present invention relates to compounds of Formula I.

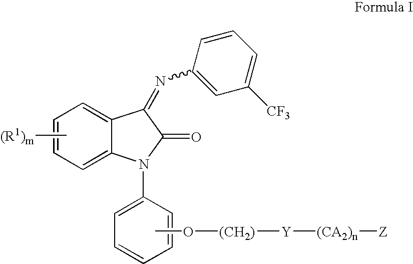

wherein each R¹ is independently straight chained or branched C₁-C₄ alkyl, straight chained or branched C₁-C₄ alkoxy, CN, F, Cl, Br or I;

wherein each A is independently H, hydroxyl or straight chained or branched C₁-C₄ alkyl;

wherein Y is —CH═CH—, —(CA₂)ₜ(NR⁵)(CH₂)— or null;

wherein Z is —N(R²)(R³),

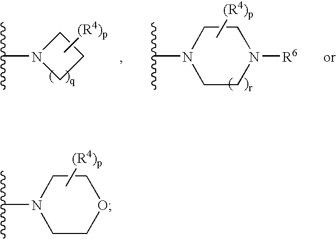

wherein R² is H, straight chained or branched C₁-C₄ dialkyl ether or straight chained or branched C₁-C₇ alkyl, wherein the C₁-C₇ alkyl may be substituted with CN or hydroxyl;

wherein R³ is H, straight chained or branched C₁-C₄ dialkyl ether or straight chained or branched C₁-C₇ alkyl, wherein the C₁-C₇ alkyl may be substituted with CN or hydroxyl or

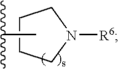

wherein each R⁴ is independently straight chained or branched C₁-C₄ dialkyl ether or straight chained or branched C₁-C₄ alkyl, wherein the C₁-C₄ alkyl may be substituted with CN or hydroxyl;

wherein R⁵ is H or straight chained or branched C₁-C₄ alkyl;

wherein R⁶ is H or straight chained or branched C₁-C₄ alkyl;

wherein m is an integer from 0 to 4 inclusive;

wherein n is an integer from 1 to 5 inclusive;

wherein p is an integer from 0 to 4 inclusive;

wherein q is an integer from 0 to 3 inclusive;

wherein r is 1 or 2;

wherein s is 1 or 2; and

wherein t is an integer from 1 to 3 inclusive;

or a pharmaceutically acceptable salt thereof.

In separate embodiments of the invention, the compound is selected from one of the specific compounds disclosed in the Experimental Section.

Furthermore, the present invention provides a pharmaceutical composition comprising a therapeutically effective amount of a compound of Formula I and a pharmaceutically acceptable carrier. The present invention also provides a process for making a pharmaceutical composition comprising admixing a compound of Formula I and a pharmaceutically acceptable carrier.

Moreover, the present invention provides a method of treating a subject suffering from depression comprising administering to the subject a therapeutically effective amount of a compound of Formula I. The present invention further provides a method of treating a subject suffering from anxiety comprising administering to the subject a therapeutically effective amount of a compound of Formula I.

### DETAILED DESCRIPTION OF THE INVENTION

Definitions

In the present invention, the term “straight chained or branched C₁-C₇ alkyl” refers to a saturated hydrocarbon having from one to seven carbon atoms inclusive. Examples of such substituents include, but are not limited to, methyl, ethyl, 1-propyl, 2-propyl, 1-butyl, 2-butyl, 2-methyl-2-propyl, 2-methyl-1-propyl, n-pentyl and 2-methylhexyl. Similarly, the term “straight chained or branched C₁-C₄ alkyl” refers to a saturated hydrocarbon having from one to four carbon atoms inclusive.

The term “straight chained or branched C₁-C₄ alkoxy” refers to a saturated alkoxy group having from one to four carbon atoms inclusive with the open valency on the oxygen. Examples of such substituents include, but are not limited to, methoxy, ethoxy, n-butoxy and t-butoxy.

The term “straight chained or branched C₁-C₄ dialkyl ether” refers to two C₁-C₄ alkyl groups bonded to a central oxygen atom (i.e. [C₁-C₄ alkyl]-O—[C₁-C₄ alkyl]). The alkyl groups need not be the same and the open valency is situated as one of the alkyl groups. Examples of such substituents include, but are not limited to, dimethyl ether, diethyl ether, methyl ethyl ether and t-butyl ethyl ether.

The specific compounds disclosed in the present invention are identified by their IUPAC names. The names of the compounds were generated using the program Chemistry 4-D Draw Nomenclator™ Database (Version 7.01c, ChemInnovation Software, Inc.). According to ChemInnovation Software Inc., Nomenclator™ automatically assigns systematic names to organic structures according to IUPAC nomenclature rules. Accordingly, this application discloses the aminoalkoxyphenyl indolone derivatives encompassed by Formula I in accordance with IUPAC nomenclature rules.

For illustrative purposes, and without limiting the invention, the compound of example 3d has the following structure:

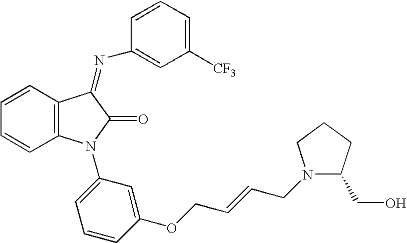

This compound is constructed from Formula I wherein m is 0; wherein Y is —CH═CH—;

wherein each A is independently H; wherein n is 1; wherein Z is

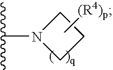

wherein q is 2; wherein p is 1 and wherein R⁴ is methyl substituted with hydroxyl.

Additionally, the invention further provides certain embodiments of the present invention that are described below.

In one embodiment, each R¹ is independently methyl, ethyl, propyl, CN, F, Cl or Br.

In one embodiment, each A is independently H, hydroxyl, methyl or ethyl.

In one embodiment, n is an integer from 1 to 3 inclusive.

In one embodiment, Z is —N(R²)(R³).

In one embodiment, R² is straight chained C₁-C₄ dialkyl ether or straight chained C₁-C₄ alkyl, wherein the C₁-C₄ alkyl may be substituted with CN or hydroxyl; and

R³ is straight chained C₁-C₄ dialkyl ether or straight chained C₁-C₄ alkyl, wherein the C₁-C₄ alkyl may be substituted with CN or hydroxyl.

In one embodiment, Y is null or —CH═CH—.

In one embodiment, Y is null.

In one embodiment, Z is

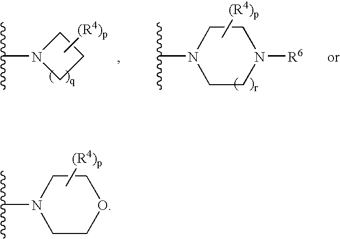

In one embodiment, each R⁴ is independently straight chained C₁-C₄ dialkyl ether or straight chained C₁-C₄ alkyl.

In one embodiment, Z is

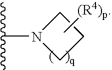

In one embodiment, Y is null and q is 2.

In one embodiment, Z is

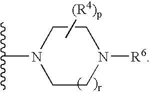

In one embodiment, Y is null and R⁶ is H or methyl.

In one embodiment, Z is

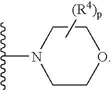

In one embodiment, Y is null and p is 0.

Pharmaceutically Acceptable Salts

The present invention also comprises salts of the present compounds, typically, pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines (for example, 8-bromotheophylline and the like). Further examples of pharmaceutically acceptable inorganic or organic acid addition salts include the pharmaceutically acceptable salts listed in S. M. Berge, et al., J. Pharm. Sci. 1977, 66, 2, the contents of which are hereby incorporated by reference.

Furthermore, the compounds of this invention may exist in unsolvated as well as in solvated forms with pharmaceutically acceptable solvents such as water, ethanol and the like. In general, the solvated forms are considered equivalent to the unsolvated forms for the purposes of this invention.

Racemic forms may be resolved into the optical antipodes by known methods, for example, by separation of diastereomeric salts thereof with an optically active acid, and liberating the optically active amine compound by treatment with a base. Separation of such diastereomeric salts can be achieved, e.g. by fractional crystallization. The optically active acids suitable for this purpose may include, but are not limited to d- or l-tartaric, madelic or camphorsulfonic acids. Another method for resolving racemates into the optical antipodes is based upon chromatography on an optically active matrix. The compounds of the present invention may also be resolved by the formation and chromatographic separation of diastereomeric derivatives from chiral derivatizing reagents, such as, chiral alkylating or acylating reagents, followed by cleavage of the chiral auxiliary. Any of the above methods may be applied either to resolve the optical antipodes of the compounds of the invention per se or to resolve the optical antipodes of synthetic intermediates, which can then be converted by methods described herein into the optically resolved final products which are the compounds of the invention.

Additional methods for the resolution of optical isomers, known to those skilled in the art, may be used. Such methods include those discussed by J. Jaques, A. Collet and S. Wilen in Enantiomers, Racemates, and Resolutions, John Wiley and Sons, New York 1981. Optically active compounds were also prepared from optically active starting materials.

The invention also encompasses prodrugs of the present compounds, which on administration undergo chemical conversion by metabolic processes before becoming pharmacologically active substances. In general, such prodrugs will be functional derivatives of the compounds of Formula I which are readily convertible in vivo into the required compound of Formula I. Conventional procedures for the selection and preparation of suitable prodrug derivatives are described in Design of Prodrugs, ed. H. Bundgaard, Elsevier, 1985.

Pharmaceutical Compositions

The present invention further provides a pharmaceutical composition comprising a therapeutically effective amount of a compound of Formula I and a pharmaceutically acceptable carrier. The present invention also provides a pharmaceutical composition comprising a therapeutically effective amount of one of the specific compounds disclosed in the Experimental Section and a pharmaceutically acceptable carrier.

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as oral, rectal, nasal, pulmonary, topical (including buccal and sublingual), transdermal, intracisternal, intraperitoneal, vaginal and parenteral (including subcutaneous, intramuscular, intrathecal, intravenous and intradermal) routes. It will be appreciated that the route will depend on the general condition and age of the subject to be treated, the nature of the condition to be treated and the active ingredient.

Pharmaceutical compositions for oral administration include solid dosage forms such as capsules, tablets, dragees, pills, lozenges, powders and granules. Where appropriate, the compositions may be prepared with coatings such as enteric coatings or they may be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include, but are not limited to, suppositories, sprays, ointments, creams, gels, inhalants, dermal patches and implants.

Typical oral dosages range from about 0.001 to about 100 mg/kg body weight per day. Typical oral dosages also range from about 0.01 to about 50 mg/kg body weight per day. Typical oral dosages further range from about 0.05 to about 10 mg/kg body weight per day. Oral dosages are usually administered in one or more dosages, typically, one to three dosages per day. The exact dosage will depend upon the frequency and mode of administration, the sex, age, weight and general condition of the subject treated, the nature and severity of the condition treated and any concomitant diseases to be treated and other factors evident to those skilled in the art.

The formulations may also be presented in a unit dosage form by methods known to those skilled in the art. For illustrative purposes, a typical unit dosage form for oral administration may contain from about 0.01 to about 1000 mg, from about 0.05 to about 500 mg, or from about 0.5 to about 200 mg.

For parenteral routes such as intravenous, intrathecal, intramuscular and similar administration, typical doses are in the order of half the dose employed for oral administration.

The present invention also provides a process for making a pharmaceutical composition comprising admixing a therapeutically effective amount of a compound of Formula I and a pharmaceutically acceptable carrier. In an embodiment of the present invention the compound utilized in the aforementioned process is one of the specific compounds disclosed in the Experimental Section.

The compounds of this invention are generally utilized as the free substance or as a pharmaceutically acceptable salt thereof. One example is an acid addition salt of a compound having the utility of a free base. When a compound of Formula I contains a free base such salts are prepared in a conventional manner by treating a solution or suspension of a free base of Formula I with a molar equivalent of a pharmaceutically acceptable acid. Representative examples of suitable organic and inorganic acids are described above.

For parenteral administration, solutions of the compounds of Formula I in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The compounds of Formula I may be readily incorporated into known sterile aqueous media using standard techniques known to those skilled in the art.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of Formula I and a pharmaceutically acceptable carrier are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and optionally a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatin capsule in powder or pellet form or it may be in the form of a troche or lozenge. The amount of solid carrier will vary widely but will range from about 25 mg to about 1 g per dosage unit.

If a liquid carrier is used, the preparation may be in the form of a syrup, emulsion, soft gelatin capsule or sterile injectable liquid such as an aqueous or non-aqueous liquid suspension or solution.

Treatment of Disorders

As mentioned above, the compounds of Formula I are ligands at the GAL₃ receptor. The present invention provides a method of treating a subject suffering from depression and/or anxiety which comprises administering to the subject a therapeutically effective amount of a compound of this invention. This invention further provides a method of treating a subject suffering from major depression and/or anxiety which comprises administering to the subject a therapeutically effective amount of a compound of this invention. In an embodiment of this invention, the subject is a human being.

The invention will be better understood from the Experimental Details which follow. However, one skilled in the art will readily appreciate that the specific methods and results discussed therein are merely illustrative of the invention as described more fully in the claims which follow thereafter. Furthermore, the variables depicted in Schemes 1-5 are consistent with the variables recited in the Summary of the Invention.

In the Experimental Section, standard acronyms are used. Examples of such acronyms include TLC (thin layer chromatography); DMF (N,N-Dimethylformamide); TEA (Triethylamine); TBDMS (t-butyl dimethylsilyl); TBAF (Tetrabutyl Ammonium Fluoride); THF (Tetrahydrofuran); rt (room temperature); h (hour); and min (minutes). Furthermore, in certain instances, the methods of preparing the compounds of the invention are described generally by referring to representative reagents such as bases or solvents. The particular reagent identified is representative but is not inclusive and does not limit the invention in any way. For example, representative bases include but are not limited to K₂CO₃, TEA or DIPEA (Diisopropylethylamine).

It may be necessary to incorporate protection and deprotection strategies for substitutents such as amino, amido, carboxylic acid and hydroxyl groups in the synthetic methods described below to form the compounds of Formula I. Methods for protection and deprotection of such groups are well known in the art, and may be found in T. Green, et al., Protection Groups in Organic Synthesis, 1991, 2^{nd} Edition, John Wiley & Sons, New York.

Experimental Section

Methods of Preparing the Compounds of Formula I

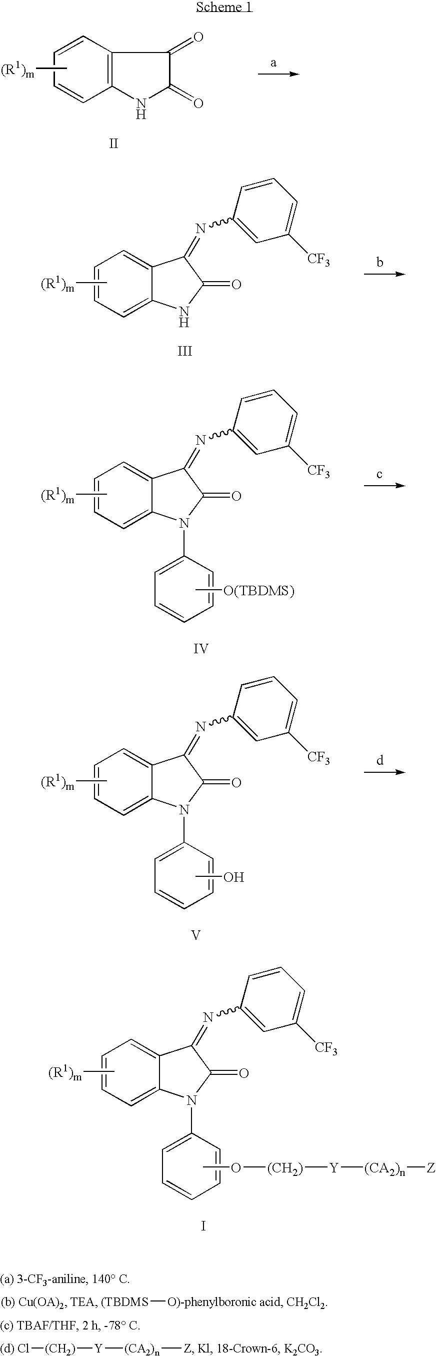

The compounds of Formula I may be synthesized according to the procedures described in Scheme 1. Isatins of Formula II are commercially available or may be synthesized according to procedures known to one skilled in the art. The intermediate of Formula III is coupled with an O-protected hydroxyphenylboronic acid that is deprotected with TBAF to afford the compounds of Formula V. O-alkylation of this intermediate provides compounds of the invention.

Substituted isatins of Formula II may be synthesized according to the procedures described in the following references: S. Garden, et al., Syn. Comm. 1998, 28, 1679-1689; G. Coppola, J. Het. Chem., 1987, 24, 1249-1251; B. Hess, et al., J. Het. Chem., 1971, 8, 161; and W. Bryant, et al, Syn. Comm., 1993, 23, 1617-1625.

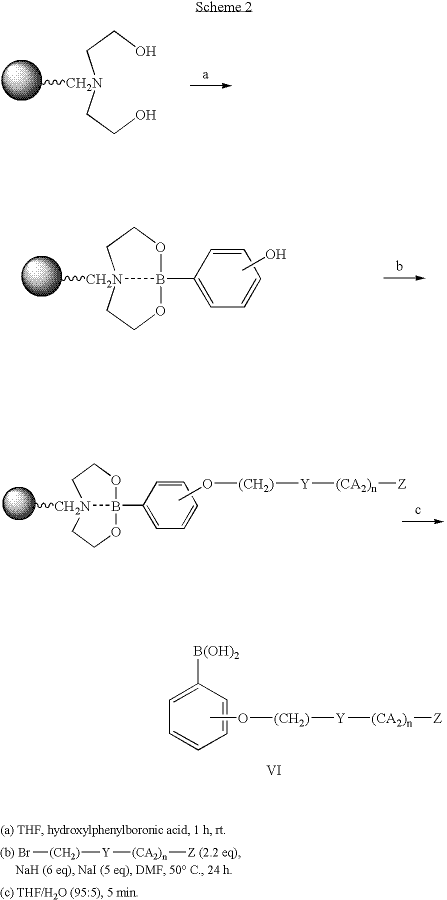

The intermediates of Formula VI, used as starting materials in Scheme 3, are prepared according to the solid-phase synthesis outlined in Scheme 2. General information regarding the solid-phase synthesis of phenylboronic acids is described in D. Hall, et al., J. Org. Chem., 2002, 67, 3-15.

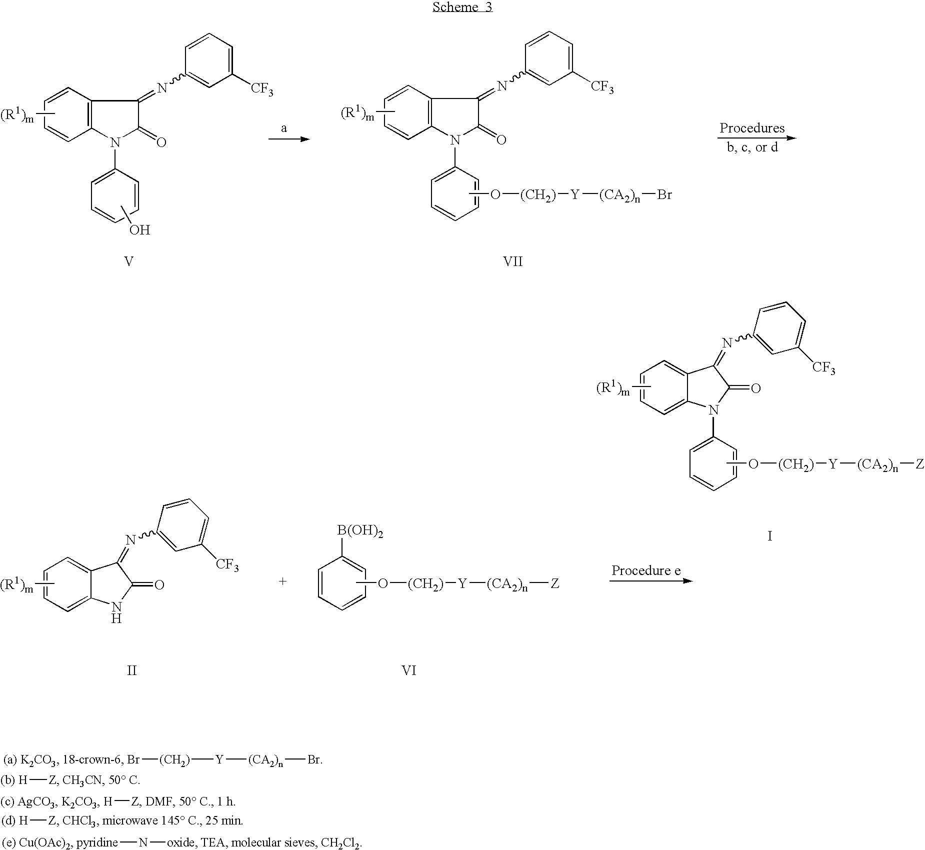

Alternatively, the compounds of Formula I may be prepared as described in Scheme 3. The intermediate of Formula VII may be treated under conditions set forth in procedures b, c and d. Additionally, the compounds of the invention may be synthesized via coupling of the compounds of Formula II and VI using the conditions described in procedure e.

The reagents, Br—(CH₂)—Y—(CA₂)ₙ—Br, may be synthesized according to the procedures described in the following references: N. P. Volynskii, et al., Ser. Khim., 1979, 1077; H. Veith, et al., Liebigs Ann., 1997, 2, 391-394; A. Thurkauf, et al., J. Org. Chem., 1987, 52, 5466-5467.; B. T. Nguyen, et al., J. Org. Chem., 1986, 51, 2206-2210; and Chiappe, C. et al. Org. Lett., 2001, 3, 1061-1063.

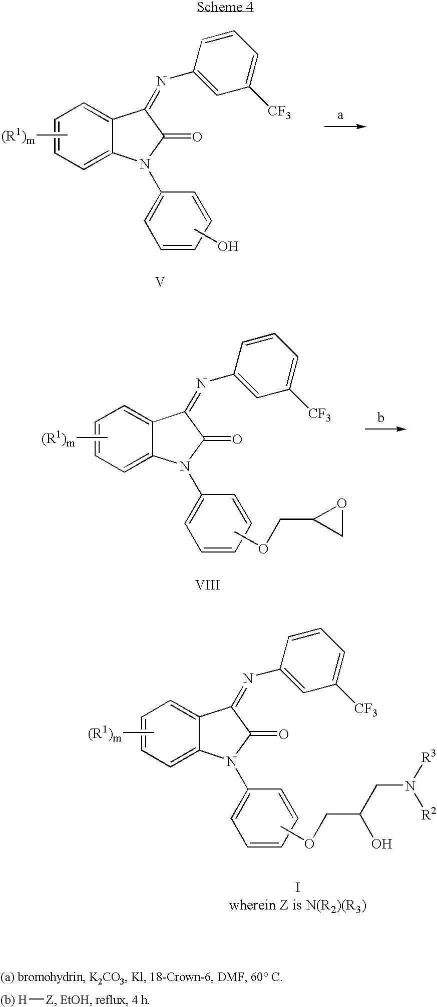

Furthermore, the compounds of Formula I, wherein one A is —OH and Z is —N(R²)(R³), may be synthesized according to the procedures described in Scheme 4. This involves the opening of the epoxide intermediate of Formula VIII with HN(R²)(R³) in EtOH. In general, to a stirred solution of aryloxyepoxide (0.1-0.2 mmol) in EtOH (5 mL), is added a slight excess of the primary or secondary amine at rt. The resulting solution is refluxed for 4 h. Concentration, followed by purification via preparative TLC affords the desired β-aryloxy aminoalcohol derivatives.

Analogs of epibromhydrin may be synthesized according to the procedures described in the following references: F. Lakner, et al., J. Org. Chem., 1996, 61, 3923-3925; R. Paul, et al., Bull. Soc. Chim. Fr., 1945, 12, 827; T. Murai, et al., J. Am. Chem. Soc., 1984, 106, 6093-6095; S. Hu, et al., Tetrahedron Lett., 1999, 40, 1641-1644; J. Aebi, et al., Ann. Chem., 1983, 2114; and M. Mitani, J. Chem. Res. Synop., 1993, 7, 249.

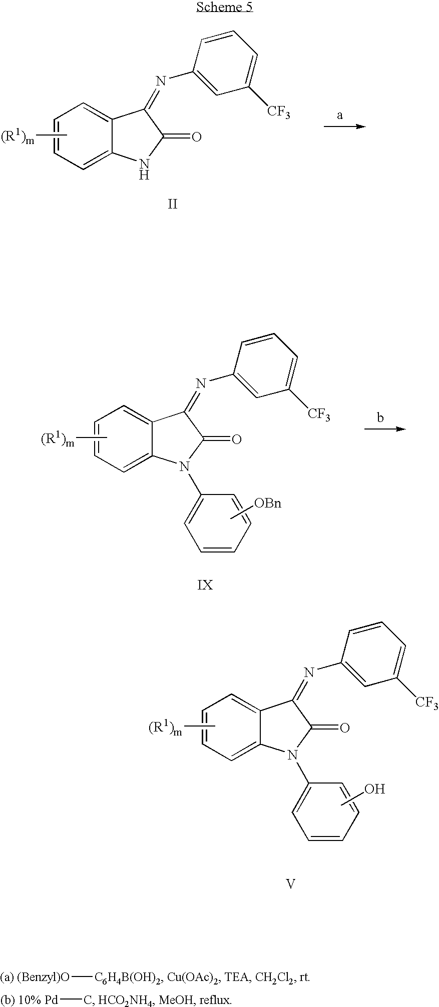

Alternatively, the compounds of Formula V, used as an advanced intermediate in Schemes 1, 3 and 4, may be synthesized according to the procedures described in Scheme 5.

General Methods:

Anhydrous solvents were purchased from Aldrich Chemical Company and used as received. The NMR spectra were measured on a Bruker Avance 400 spectrometer with CDCl₃ as the solvent with tetramethylsilane as the internal standard unless otherwise noted. Chemical shifts (δ) are expressed in ppm, coupling constants (J) are expressed in Hz, and splitting patterns are described as follows: s=singlet; d=doublet; t=triplet; q=quartet; br=broad; m=multiplet; dd=doublet of doublets; dt=doublet of triplets; td=triplet of doublets; dm=doublet of multiplets. Unless otherwise noted, mass spectra were obtained using electrospray ionization (ESMS, Micromass Platform II or Quattro Micro) and (M+H)⁺ is reported. Thin-layer chromatography (TLC) was carried out on glass plates pre-coated with silica gel 60 F₂₅₄ (0.25 mm, EM Separations Tech.). Preparative TLC was carried out on glass sheets pre-coated with silica gel GF (2 mm, Analtech). Flash column chromatography was performed on Merck silica gel 60 (230-400 mesh). Microwave—reactions were performed in a Personal Synthesizer® microwave.

Preparation of Intermediates

The compounds of Formula III were synthesized as follows:

3-{[3-(trifluoromethyl)phenyl]azamethylene}-1H-benzo[d]azolin-2-one:

Isatin (31.0 g, 0.210 mol) was combined with 3-(trifluoromethyl)aniline (132 mL, 170 g, 1.05 mol) and heated at 140° C. for 6 h. The reaction was cooled to rt, and the crystals were collected by filtration and washed with cold methanol, yielding the desired product (57.1 g, 95%). ESMS m/e: 290 (M+H)⁺.

The following substituted 3-{[3-(trifluoromethyl)phenyl]azamethylene}-1H-benzo[d]azolin-2-ones were prepared analogously:

5-fluoro-3-{[3-(trifluoromethyl)phenyl]azamethylene}-1H-benzo[d]azolidin-2-one

ESMS m/e: 309 (M+H)⁺;

5,7-dimethyl-3-{[3-(trifluoromethyl)phenyl]azamethylene}-1H-benzo[d]azolidin-2-one:

ESMS m/e: 319 (M+H)⁺;

2-oxo-3-{[3-(trifluoromethyl)phenyl]azamethylene}-1H-benzo[d]azolidine-6-carbonitrile:

ESMS m/e: 316 (M+H)⁺; and

6-Bromo-5-methyl-3-{[3-(trifluoromethyl)phenyl]azamethylene}-1H-benzo[d]azolidin-2-one

ESMS m/e: 383, 385 (M+H)⁺.

The compounds of Formulas IV, V and VII were synthesized as follows:

1-[3-(1,1,2,2-Tetramethyl-1-silapropoxy)phenyl]-3-{[3-(trifluoromethyl)phenyl]azamethylene}benzo[d]azolin-2-one:

A mixture of 3-{[3-(Trifluoromethyl)phenyl]azamethylene}-1H-benzo[d]azolin-2-one (3.19 g, 0.011 mol), 3-(TBDMS-O)-phenylboronic acid (2.78 g, 0.013 mol), and Cu(OAc)₂ (1.79 g, 0.012 mmol) in CH₂Cl₂ (300 mL) was stirred at rt for 5 min. TEA (4.59 mL) was added dropwise. The reaction was stirred at rt overnight. The reaction was diluted with CH₂Cl₂ and washed with saturated EDTA solution (2×) and water. After drying over Na₂SO₄, the organic solvent was removed in vacuo. The crude product was purified by silica gel chromatography, eluting with CHCl₃/2M NH₃ in MeOH (49:1), giving the desired product (3.36 g, 62% yield). ESMS m/e: 497 (M+H)⁺.

1-(3-Hydroxyphenyl)-3-{[3-(trifluoromethyl)phenyl]azamethylene}benzo[d]azolin-2-one:

1-[3-(1,1,2,2-Tetramethyl-1-silapropoxy)phenyl]-3-{[3(trifluoromethyl)phenyl]azamethylene}benzo[d]azolin-2-one (336 mg, 0.67 mmol) in anhydrous THF (15 mL) was cooled to −78° C. and then TBAF in THF (1.0 M, 0.677 mL) was added dropwise. The reaction progress was monitored by TLC and typically was complete within 1 h. Upon completion, the mixture was allowed to warm up to rt. The solvent was removed in vacuo and the product was re-dissolved in CH₂Cl₂, washed with water several times and dried over Na₂SO₄. Solvent removal gave of the desired product (218 mg, 85%). ESMS m/e: 383 (M+H)⁺.

1-[4-(3-Bromopropoxy)phenyl]-3-{[3-(trifluoromethyl)phenyl]azamethylene}benzo[d]azolin-2-one:

1-(4-hydroxyphenyl)-3-{[3-(trifluoromethyl)phenyl]azamethylenebenzo[d]azolin-2-one (200 mg, 0.52 mmol) was combined with K₂CO₃ (72 mg, 0.52 mmol), KI (86 mg, 0.52 mmol), and 1,3-dibromopropane (315 mg, 1.56 mmol) in anhydrous DMF (1 mL). The reaction was heated at 60° C. under argon for 16 h. Upon cooling the reaction to rt, EtOAc (15 mL) was added, and the mixture was washed with water (2×8 mL) and brine (1×8 mL). After drying the EtOAc layer over MgSO₄, the mixture was filtered and concentrated in vacuo. The crude product residue was loaded onto a silica gel plug and purified by silica gel column chromatography, eluted with hexane/EtOAc/TEA (90:10:1). The first orange band was collected and yielded the desired product (115 mg, 45% yield). ESMS m/e: 503 (M+H)⁺.

The following compounds were prepared analogously:
1-[3-(3-Bromopropoxy)phenyl]-3-{[3-(trifluoromethyl)phenyl]azamethylene}benzo[d]azolin-2-one;
1-[3-(4-Bromobutoxy)phenyl]-3-{[3-(trifluoromethyl)phenyl]azamethylene}benzo[d]azolin-2-one; and
1-[3-(5-Bromopentoxy)phenyl]-3-{[3-(trifluoromethyl)phenyl]azamethylene}benzo[d]azolin-2-one.

The compounds of Formula VI were synthesized as follows:

PS DEAM resin (5.00 g, loading 1.66 mmol g⁻¹) and anhydrous THF (60 mL) was added to a polypropylene fritted column. 3-Hydroxyphenylboronic acid (1.47 g, 10.7 mmol) was added and the column sealed and placed on a spinning wheel for 4 h. After 4 h, the THF was drained and the resin washed with anhydrous THF (2×20 mL). The resin was then dried under vacuum to afford the resin bound phenylboronic acid.

The resin-bound phenylboronic acid, NaI (5 eq) and 3-diethylaminopropyl chloride (2.2 eq) were placed in oven-dried pyrex jar under argon. Anhydrous DMF (50 mL) was added and the mixture was stirred on a circular shaker at 50° C. for 30 min. NaH (5 eq) was added and the mixture was allowed to stir at 50° C. After 24 h, the mixture was filtered thorough a polypropylene fritted column under argon and the resin washed with anhydrous DMF (3×20 mL), anhydrous CH₂Cl₂ (3×20 mL) and anhydrous THF (3×20 mL). Cleavage of the product was achieved by stirring the resin in THF/water (95:5, 60 mL) for 5 min on a spinning wheel. The solvent mixture was collected by filtration and concentrated in vacuo, yielding the desired material (1.14 g).

The intermediate of Formula VIII was synthesized as follows:

1-[3-(Oxiran-2-ylmethoxy)phenyl]-3-{[3-(trifluoromethyl)phenyl]azamethylene}benzo[d]azolidin-2-one:

Epibromohydrin (587 mg, 4.32 mmol) was added to a solution of N-(3-hydroxyphenyl)-3′-(trifluoromethylphenyl)iminoisatin (1.1 g, 2.88 mmol), KI (239 mg, 1.44 mmol), K₂CO₃ (596 g, 4.32 mmol and 18-Crown-6 (380 mg, 1.43 mmol) in DMF (25 mL) at rt. The mixture was heated at 80° C. under argon atmosphere for 16 h. After 16 h, the reaction mixture was quenched with water. The product was extracted with EtOAc and the EtOAc layer was washed successively with water and brine. Purification by column chromatography on silica gel using EtOAc/hexanes (1:1) afforded 1-[3-(Oxiran-2-ylmethoxy)phenyl]-3-{[3-(trifluoromethyl)phenyl]azamethylene}benzo[d]azolidin-2-one as a pale yellow solid (380 mg, 30%). ESMS m/e: 439 (M +H)⁺.

The following two compounds were prepared analogously:

(R)-1-[3-(Oxiran-2-ylmethoxy)phenyl]-3-{[3-(trifluoromethyl)phenyl]azamethylene}benzo[d]azolidin-2-one:

ESMS m/e: 439 (M+H)⁺. (R)-epichlorohydrin was used in place of epibromohydrin.

(S)-1-[3-(Oxiran-2-ylmethoxy)phenyl]-3-{[3-(trifluoromethyl)phenyl]azamethylene}benzo[d]azolidin-2-one:

ESMS m/e: 439 (M +H)⁺. (S)-epichlorohydrin was used in place of epibromohydrin.

The intermediate of Formula IX was synthesized as follows:

1-[2-(Phenylmethoxy)phenyl]-3-{[3-(trifluoromethyl)phenyl]azamethylene}benzo[d]azolidin-2-one:

Triethylamine (7.4 mL, 54 mmol) was added to a stirring solution of 3-{[3-(trifluoromethyl)phenyl]azamethylene}-1H-benzo[d]azolin-2-one (5.3 g, 18 mmol), copper acetate (3.59 g, 19.8 mmol) and 2-benzyloxyphenylboronic acid (5.0 g, 22 mmol) in dichloromethane (600 mL) at rt under nitrogen atmosphere. After stirring overnight at rt, water was added and the layers were separated. The organic portion was washed with a saturated solution of ethylenediaminetetraacetic acid in water. Purification was performed on silica gel chromatography, eluting with 5% 2M NH₃ in MeOH/CHCl₃ (1:19), giving 1-[2-(phenylmethoxy)phenyl]-3-{[3-(trifluoromethyl)phenyl]azamethylene}benzo[d]azolidin-2-one as pale yellow color foam (2.0 g, 25%). ESMS m/e: 473 (M +H)⁺.

The intermediate of Formula V was synthesized as follows:

1-[2-(Hydroxy)phenyl]-3-{[3-(trifluoromethyl)phenyl]azamethylene}benzo[d]azolidin-2-one:

10% Pd—C (101 mg) was added to a stirring solution of 1-[2-(benzyloxy)phenyl]-3-{[3-(trifluoromethyl)phenyl]azamethylene}benzo[d]azolidin-2-one (630 mg, 1.33 mmol) and ammonium formate (168 mg, 2.66 mmol) in methanol (70 mL) at rt. The resulting mixture was then heated at reflux for 2 h. The reaction mixture was filtered through a pad of celite and then the solvent was removed, leaving a crude product. Purification was performed on silica gel chromatography, giving 1-[2-(hydroxy)phenyl]-3-{[3-(trifluoromethyl)phenyl]azamethylene}benzo[d]azolidin-2-one as dark brown color foam (170 mg, 33%). ESMS m/e: 383 (M+H)⁺.

TABLE 1
Compounds List

| Example | |
|---|---|
| No. | Structure |
| 1a | 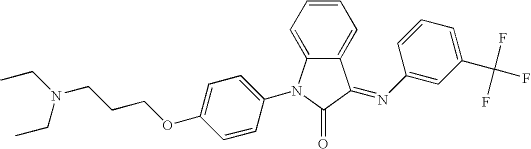 |
| 1b | 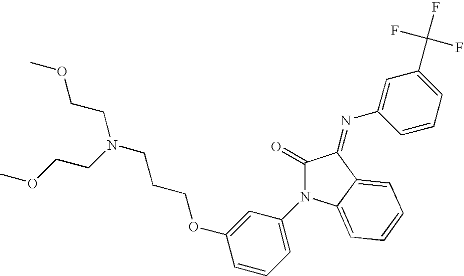 |
| 1c | 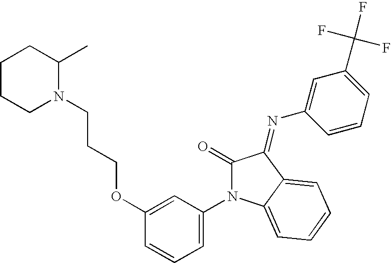 |
| 1d | 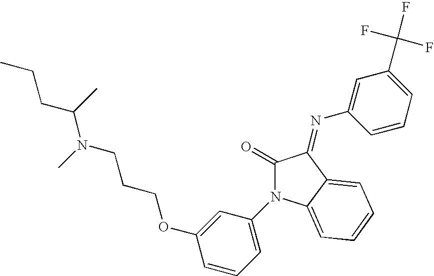 |
| 1e | 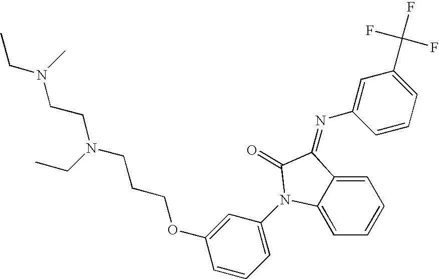 |
| 1f | 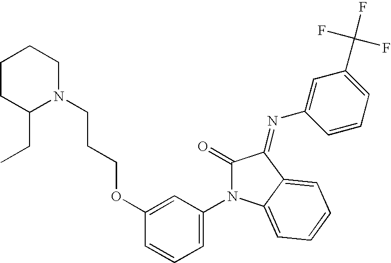 |
| 1g | 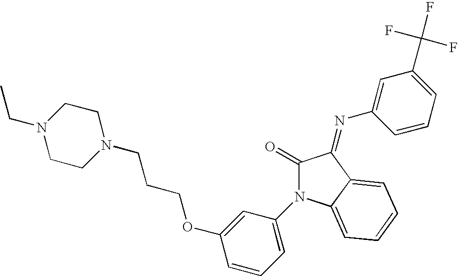 |
| 1h | 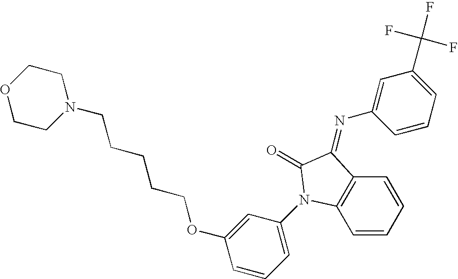 |
| 1i | 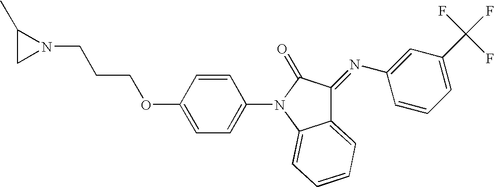 |
| 1j | 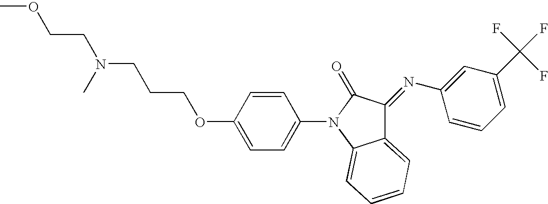 |
| 1k | 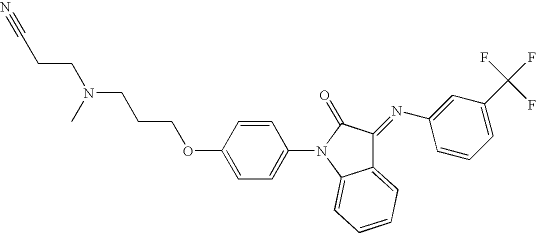 |
| 1l | 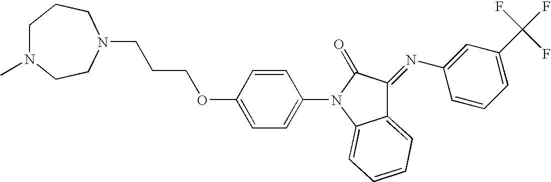 |
| 1m | 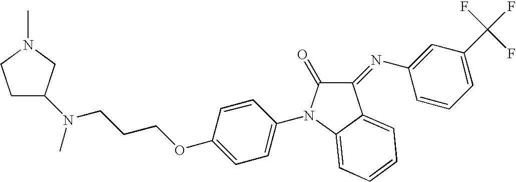 |
| 1n | 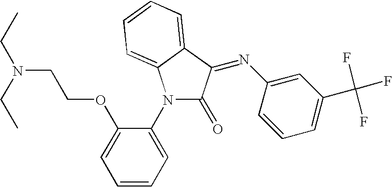 |
| 1o | 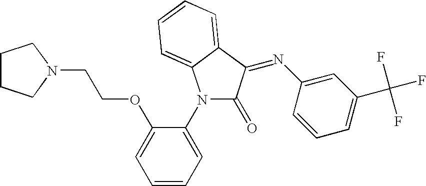 |
| 1p | 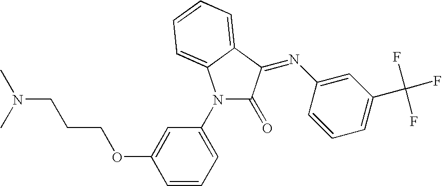 |
| 1q | 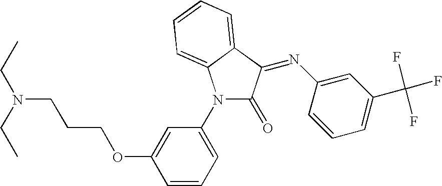 |
| 1r | 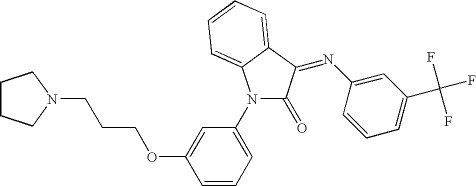 |
| 1s | 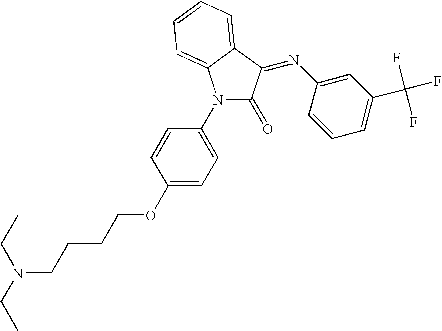 |
| 2a | 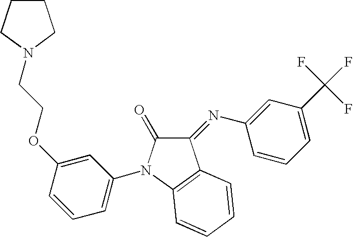 |
| 2b | 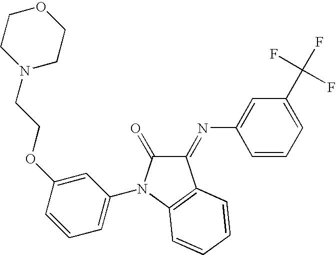 |
| 2c | 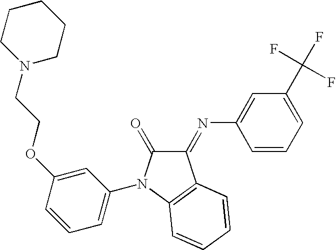 |
| 2d | 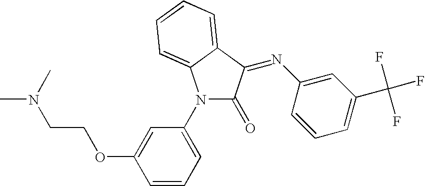 |
| 2e | 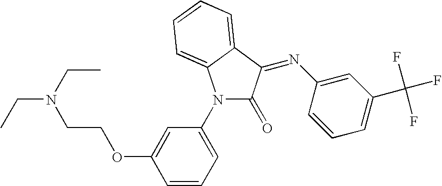 |
| 2f | 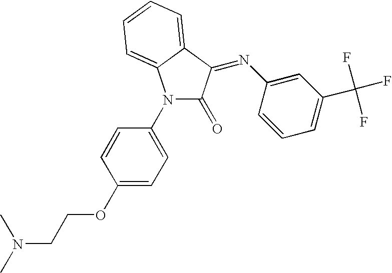 |
| 2g | 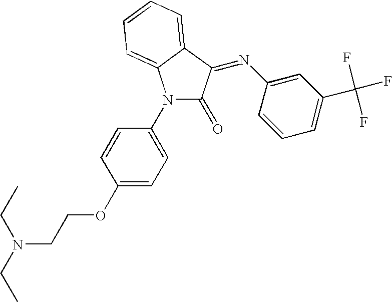 |
| 3a | 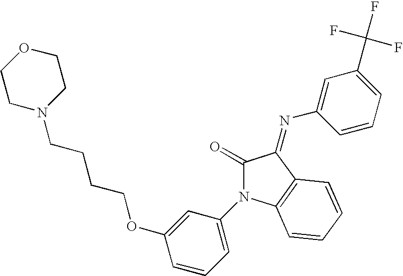 |
| 3b | 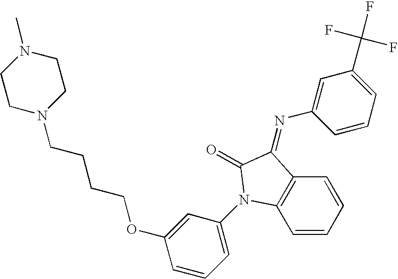 |
| 3c | 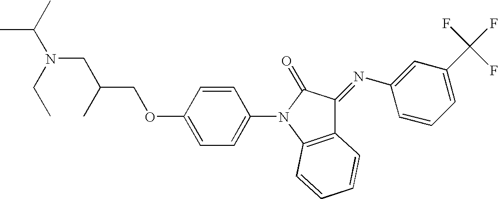 |
| 3d | 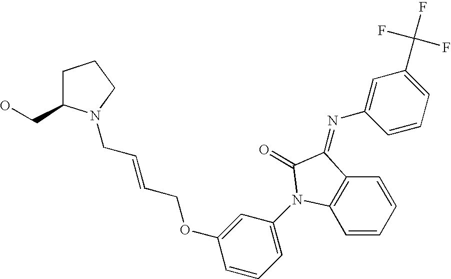 |
| 4a | 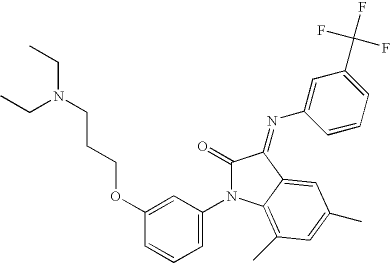 |
| 4b | 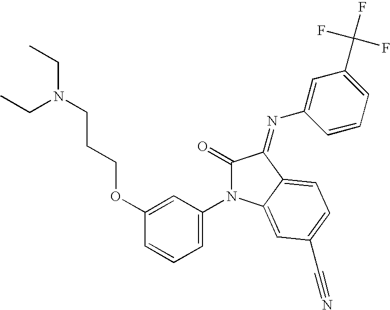 |
| 4c | 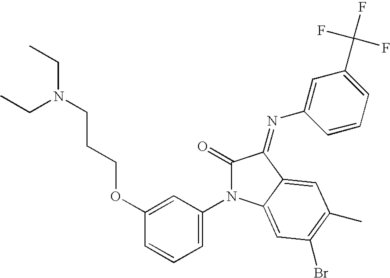 |
| 5a | 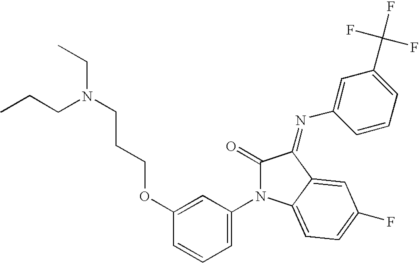 |
| 5b | 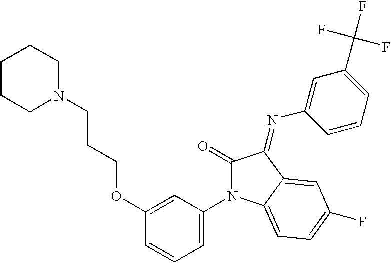 |
| 5c | 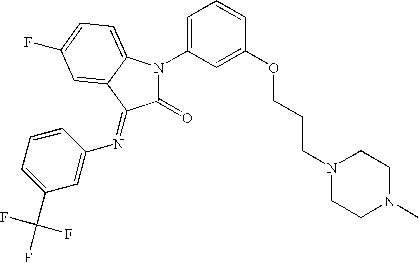 |
| 6a | 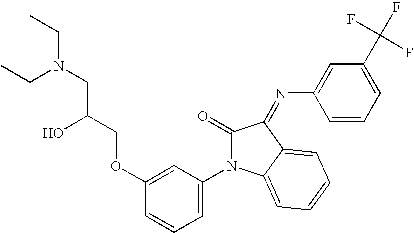 |
| 6b | 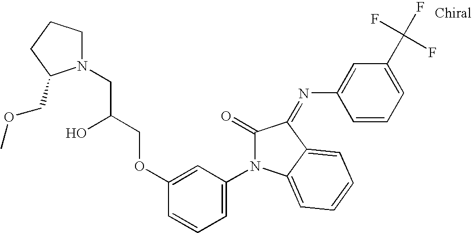 |
| 6c | 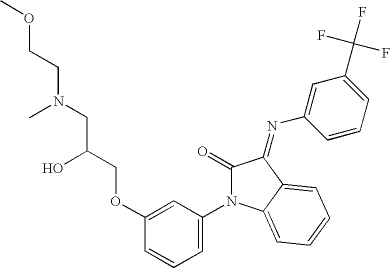 |
| 6d | 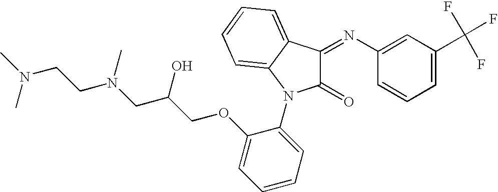 |
| 6e | 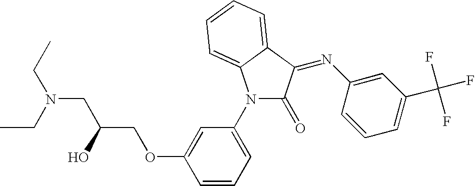 |
| 6f | 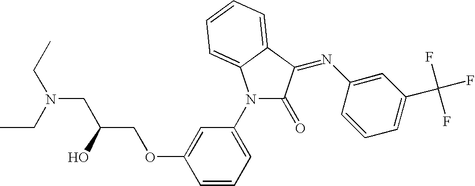 |

The following compounds were prepared according to procedure b as described in Scheme 3.

### EXAMPLE 1a

### 1-{4-[3-(Diethylamino)propoxy]phenyl}-3-{[3-(trifluoromethyl)phenyl]azamethylene}benzo[d]azolidin-2-one

1-[4-(3-Bromopropoxy)phenyl]-3-{[3-(trifluoromethyl)phenyl]azamethylene}benzo[d]azolin-2-one (150 mg, 0.300 mmol) was combined with diethylamine (55 mg, 0.75 mmol) and molecular sieves in anhydrous CH₃CN (5 mL) and heated at 60° C. under argon atmosphere for 12 h. Upon cooling to rt, the reaction mixture was concentrated in vacuo. The remaining crude residue was re-dissolved in EtOAc and washed with water and then with brine. After drying the final EtOAc layer over MgSO₄, the organic layer was filtered and concentrated in vacuo, giving a crude product. The product was purified by preparative TLC, eluting with EtOAc/hexane/TEA (70:30:1), giving the desired product (22 mg, 34%). ¹H NMR δ 7.57 (2H, m), 7.33 (3H, t, J=11), 7.26 (2H, q, J=9.0), 7.08 (2H, d, J=9.7), 6.8 (2H, t, J=5.3), 6.59 (1H, d, J=9.0), 4.08 (2H, t, J=7.7), 2.68 (2H, t, J=9), 2.6 (4H, q, J=10), 1.99 (2H, q, J=8.5), 1.06 (6H, t, J=10); ESMS m/e: 496 (M+H)⁺.

The following compounds were prepared analogously:

### EXAMPLE 1b

### 1-(3-{3-[Bis(2-methoxyethyl)amino]propoxy}phenyl)-3-{[3(trifluoromethyl)phenyl]azamethylene}benzo[d]azolidin-2-one

ESMS m/e: 556 (M+H)⁺.

### EXAMPLE 1c

### 1-{3-[3-((2S)-2-Methylpiperidyl)propoxy]phenyl}-3-{[3-(trifluoromethyl)phenyl]azamethylene}benzo[d]azolidin-2-one

ESMS m/e: 522 (M+H)⁺.

### EXAMPLE 1d

### 1-(3-{3-[Methyl(methylbutyl)amino]propoxyphenyl)-3-{[3-(trifluoromethyl)phenyl]azamethylene}benzo[d]azolidin-2-one

ESMS m/e: 524 (M+H)⁺.

### EXAMPLE 1e

### 1-[3-(3-{Ethyl[2-(ethylmethylamino)ethyl]amino}propoxy)phenyl]-3-{[3-(trifluoromethyl)phenyl]azamethylene}benzo[d]azolidin-2-one

ESMS m/e: 553 (M+H)⁺.

### EXAMPLE 1f

### 1-3-[3-(2-Ethylpiperidyl)propoxy]phenyl}-3-{[3-(trifluoromethyl)phenyl]azamethylene}benzo[d]azolidin-2-one

ESMS m/e: 536 (M+H)⁺.

### EXAMPLE 1g

### 1-3-[3-(4-Ethylpiperazinyl)propoxy]phenyl}-3-{[3-(trifluoromethyl)phenyl]azamethylene}benzo[d]azolidin-2-one

ESMS m/e: 537 (M+H)⁺.

### EXAMPLE 1h

### 1-[3-(5-Morpholin-4-ylpentyloxy)phenyl]-3-{[3-(trifluoromethyl)phenyl]azamethylene}benzo[d]azolin-2-one

ESMS m/e: 538 (M+H)⁺.

### EXAMPLE 1i

### 1-{4-[3-(2-Methylaziridinyl)propoxy]phenyl}-3-{[3-(trifluoromethyl)phenyl]azamethylene}benzo[d]azolidin-2-one

ESMS m/e: 480 (M+H)⁺.

### EXAMPLE 1j

### 1-(4-{3-[(2-Methoxyethyl)methylamino]propoxy}phenyl)-3-{[3-(trifluoromethyl)phenyl]azamethylene}benzo[d]azolidin-2-one

ESMS m/e: 512 (M+H)⁺.

### EXAMPLE 1k

### 3-(Methyl{3-[4-(2-oxo-3-{[3-(trifluoromethyl)phenyl]azamethylene}benzo[d]azolidinyl)phenoxy]propyl}amino)propanenitrile

ESMS m/e: 507 (M+H)⁺.

### EXAMPLE 1l

1-{4-[3-(4-Methyl(1,4-diazaperhydroepinyl))propoxy]phenyl}-3-{[3-(trifluoromethyl)phenyl]azamethylene}benzo[d]azolidin-2-one

ESMS m/e: 537 (M+H)⁺.

### EXAMPLE 1m

### 1-(4-{3-[Methyl(1-methylpyrrolidin-3-yl)amino]propoxy}phenyl)-3-{[3-(trifluoromethyl)phenyl]azamethylene}benzo[d]azolidin-2-one

ESMS m/e: 537 (M+H)⁺.

### EXAMPLE 1n

### 1-{2-[2-(Diethylamino)ethoxy]phenyl}-3-{[3-(trifluoromethyl)phenyl]azamethylene}benzo[d]azolidin-2-one

ESMS m/e: 482 (M+H)⁺.

### EXAMPLE 1o

### 1-[2-(2-Pyrrolidinylethoxy)phenyl]-3-{[3-(trifluoromethyl)phenyl]azamethylene}benzo[d]azolidin-2-one

ESMS m/e: 480 (M +H)⁺.

### EXAMPLE 1p

### 1-{3-[3-(Dimethylamino)propoxy]phenyl}-3-{[3-(trifluoromethyl)phenyl]azamethylene}benzo[d]azolidin-2-one

ESMS m/e: 468 (M +H)⁺.

### EXAMPLE 1q

### 1-[3-(3-Diethylamino-propoxy)-phenyl]-3-(3-trifluoromethyl-phenylimino)-1,3-dihydroindol-2-one

ESMS m/e: 496 (M +H)⁺.

### EXAMPLE 1r

### 1-[3-(3-Pyrrolidinylpropoxy)phenyl]-3-{[3-(trifluoromethyl)phenyl]azamethylene}benzo[d]azolidin-2-one

ESMS m/e: 494 (M +H)⁺.

### EXAMPLE 1s

### 1-{4-[4-(Diethylamino)butoxy]phenyl}-3-{[3-(trifluoromethyl)phenyl]azamethylene}benzo[d]azolin-2-one

ESMS m/e: 510 (M +H)⁺.

The following compounds were prepared according to the procedures described in Scheme 1.

### EXAMPLE 2a

### 1-[3-(2-Pyrrolidinylethoxy)phenyl]-3-{[3-(trifluoromethyl)phenyl]azamethylene}benzo[d]azolin-2-one

1-(3-Hydroxyphenyl)-3-{[3-(trifluoromethyl)phenyl]azamethylene}benzo[d]azolin-2-one (450 mg, 1.18 mmol) was added to K₂CO₃ (406 mg, 2.95 mmol), KI (215 mg, 1.3 mmol), and 18-crown-6 (343 mg, 1.3 mmol) in anhydrous DMF (50 mL). The reaction mixture was stirred at rt under argon atmosphere for 30 min and then (2-chloroethyl)pyrrolidine hydrochloride (401 mg, 2.36 mmol) was added. The reaction mixture was heated at 60° C. overnight under argon. The reaction mixture was passed through a pad of celite and the solvent was removed in vacuo. The crude product residue was partitioned into CH₂Cl₂ and water. The CH₂Cl₂ layer was washed with brine, dried over Na₂SO₄, and concentrated to a dark oil. Purification was carried out by chromatography on silica gel, eluting with 3-10% MeOH in CH₂Cl₂, giving 1-[3-(2-pyrrolidinylethoxy)phenyl]-3-{[3-(trifluoromethyl)phenyl]azamethylene}benzo[d]azolin-2-one (236 mg, 41%). ¹H NMR δ 7.59 (2H, m), 7.46 (1H, t, J=9.9), 7.33 (3H, m), 7.03 (2H, t, J=8.4), 6.89 (3H, m), 6.62 (1H, d, J=9.5), 4.15 (2H, t, J=7.3), 2.94 (2H, t, J=7.3), 2.65 (4H, s, br), 1.82 (4H, s, br); ESMS m/e: 480 (M+H)⁺.

The following compounds were prepared analogously:

### EXAMPLE 2b

### 1-[3-(2-Morpholin-4-ylethoxy)phenyl]-3-{[3-(trifluoromethyl)phenyl]azamethylene}benzo[d]azolin-2-one

ESMS m/e: 496 (M+H)⁺.

### EXAMPLE 2c

### 1-[3-(2-Piperidylethoxy)phenyl]-3-{[3-(trifluoromethyl)phenyl]azamethylene}benzo[d]azolin-2-one

ESMS m/e: 494 (M+H)⁺.

### EXAMPLE 2d

### 1-{3-[2-(Dimethylamino)ethoxy]phenyl}-3-{[3-(trifluoromethyl)phenyl]azamethylene}benzo[d]azolin-2-one

ESMS m/e: 454 (M+H)⁺.

### EXAMPLE 2e

### 1-{3-[2-(Diethylamino)ethoxy]phenyl}-3-{[3-(trifluoromethyl) phenyl]azamethylene}benzo[d]azolin-2-one

ESMS m/e: 482 (M+H)⁺.

### EXAMPLE 2f

### 1-{4-[2-(Dimethylamino)ethoxy]phenyl}-3-{[3-(trifluoromethyl)phenyl]azamethylene}benzo[d]azolin-2-one

ESMS m/e: 454 (M+H)⁺.

### EXAMPLE 2g

### 1-{4-[2-(Diethylamino)ethoxy]phenyl}-3-{[3-(trifluoromethyl)phenyl]azamethylene}benzo[d]azolin-2-one

ESMS m/e: 482 (M +H)⁺.

The following compounds were prepared as described in Scheme 3 using procedure d.

### EXAMPLE 3a

### 1-[3-(4-Morpholin-4-ylbutoxy)phenyl]-3-{[3-(trifluoromethyl)phenyl]azamethylene}benzo[d]azolin-2-one

Morpholine (25 mg, 0.57 mmol) was added to a solution of 1-[3-(4-bromobutoxy)phenyl]-3-{[3-(trifluoromethyl)phenyl]azamethylene}benzo[d]azolin-2-one (10 mg, 0.02 mmol) in CHCl₃ (1 mL) inside a sealed tube. The solution was heated in a microwave at 145° C. for 25 min. Additional CHCl₃ (5 mL) was added and the resulting solution was washed with cold saturated NaHCO₃ and the organic layer was dried with Na₂SO₄. The product was purified on a silica preparative plate, eluting with EtOAc/methanol (1:2), giving the desired product (3 mg, 30%). ¹H NMR δ 7.54-6.72 (11H, m), 6.56 (1H, d, J=8), 3.97-3.89 (2H, m), 3.67-3.58 (4H, m), 2.74-2.69 (2H, m, b), 2.38-2.32 (4H, m), 1.82-1.57 (4H, m); ESMS m/e: 524 (M+H)⁺.

The following compounds were prepared analogously:

### EXAMPLE 3b

### 1-{3-[4-(4-Methylpiperazinyl)butoxy]phenyl}-3-{[3-(trifluoromethyl)phenyl]azamethylene}benzo[d]azolin-2-one

ESMS m/e: 537 (M+H)⁺.

### EXAMPLE 3c

### 1-(4-{3-[Ethyl(methylethyl)amino]-2-methylpropoxy}phenyl)-3-{[3-(trifluoromethyl)phenyl]azamethylene}benzo[d]azolin-2-one

ESMS m/e: 524 (M+H)⁺.

### EXAMPLE 3d

### 1-(3-{(2E)4-[(2R)-2-(Hydroxymethyl)pyrrolidinyl]but-2-enyloxy}phenyl)-3-{[3-(trifluoromethyl)phenyl]azamethylene}benzo[d]azolin-2-one

ESMS m/e: 536 (M+H)⁺.

The following compounds were prepared as described in Scheme 3 using procedure e.

### EXAMPLE 4a

### 1-{3-[3-(Diethylamino)propoxy]phenyl}-5,7-dimethyl-3-{[3(trifluoromethyl)phenyl]azamethylene}benzo[d]azolidin-2-one

5,7-Dimethyl-3-{[3-(trifluoromethyl)phenyl]azamethylene}-1H-benzo[d]azolidin-2-one (30 mg) was combined with Cu(OAc)₂ (35 mg), 3-(3-diethylaminoproxyl)phenyl boronic acid (48 mg) in 8 mL of CH₂Cl₂ with pyridine N-oxide (10 mg) and crushed 4A° molecular sieves (100 mg). The mixture was allowed to stir at 0° C. and TEA (53 ul) added and the mixture stirred at rt for 72 h. The mixture was then diluted with 10 mL of EtOAc. The reaction mixture was then washed with water and brine. After drying the final EtOAc layer over Na₂SO₄, the solution was concentrated in vacuo. The product was purified by silica gel prep plate, eluting with 10% methanol in CH₂Cl₂, giving 1-{3-[3-(diethylamino)propoxy]phenyl}-5,7-dimethyl-3-{[3(trifluoromethyl)phenyl]azamethylene}benzo[d]azolidin-2-one (22%). ¹H NMR δ 7.55-6.35 (m, 10H), 3.94-3.80 (m, 2H), 2.72-2.57 (m, 6H), 2.25 (m, 2H), 1.8 (s, 3H), 1.55 (s, 3H), 0.96 (6H, t, J=10); ESMS m/e: 524 (M+H)⁺.

The following compounds were prepared analogously:

### EXAMPLE 4b

### 1-{3-[3-(Diethylamino)propoxy]phenyl}-2-oxo-3-{[3-(trifluoromethyl)phenyl]azamethylene}benzo[d]azolidine-6-carbonitrile

ESMS m/e: 521 (M+H)⁺.

### EXAMPLE 4c

### 1-{3-[3-(Diethylamino)propoxy]phenyl}-6-bromo-5-methyl-3-{[3-(trifluoromethyl)phenyl]azamethylene}benzo[d]azolidin-2-one

ESMS m/e: 588 and 590 (M+H)⁺.

The following compounds were prepared as described in Scheme 3 using procedure c.

### EXAMPLE 5a

### 1-{3-[3-(Ethylpropylamino)propoxy]phenyl}-5-fluoro-3-{[3-(trifluoromethyl)phenyl]azamethylene}benzo[d]azolidin-2-one

1-[3-(3-Bromopropoxy)phenyl]-5-fluoro-3-{[3-(trifluoromethyl)phenyl]azamethylene}benzo[d]azolidin-2-one (0.038 mmol) was combined with AgCO₃ (0.028 mmol), K₂CO₃ (0.038 mmol) and ethylpropylamine (0.076 mmol) in anhydrous DMF (1 mL). The reaction was heated at 55° C. under argon for 24 h. Upon cooling the reaction to rt, EtOAc (10 mL) was added, and the mixture was washed with water (2×10 mL) and brine (1×10 mL). After drying the EtOAc layer over MgSO₄, the solution was filtered and concentrated in vacuo. The crude product was purified by preparative TLC, eluting with MeOH/CH₂Cl₂ (1:9) giving the desired product. ¹H NMR (CD₃OD) δ 7.68-6.68 (m, 10H), 6.15-6.08 (m, 1H), 4.03-3.90 (m, 2H), 2.56-2.35 (m, 6H), 1.98-1.86 (m, 2H), 1.60-1.48 (m, 6H), 1.45-1.30 (m, 2H); ESMS m/e: 528 (M+H)⁺.

The following compounds were prepared analogously:

### EXAMPLE 5b

### 5-Fluoro-1-[3-(3-piperidylpropoxy)phenyl]-3-{[3-(trifluoromethyl)phenyl]azamethylene}benzo[d]azolidin-2-one

ESMS m/e: 526 (M+H)⁺.

### EXAMPLE 5c

### 5-Fluoro-1-{3-[3-(4-methylpiperazinyl)propoxy]phenyl}-3-{[3-(trifluoromethyl)phenyl]azamethylene}benzo[d]azolidin-2-one

ESMS m/e: 541 (M+H)⁺.

The following compounds were prepared as described in Schemes 4 and 5.

### EXAMPLE 6a

### 1-{3-[3-(Diethylamino)-2-hydroxypropoxy]phenyl}-3-{[3-(trifluoromethyl)phenyl]azamethylene}benzo[d]azolidin-2-one

1-[3-(Oxiran-2-ylmethoxy)phenyl]-3-{[3-(trifluoromethyl)phenyl]azamethylene}benzo[d]azolidin-2-one (50 mg, 0.114 mmol) was dissolved in EtOH (5 mL) and diethylamine (20 mg) was added at rt. The resulting pale brown solution was heated at reflux for 4 h. Purification by preparative TLC afforded the desired product as an orange semi-solid (33 mg, 59%). ¹H NMR δ 7.63-6.76 (m, 11H), 6.59 (d, 1H, J=6.0), 4.13-3.89 (m, 3H), 2.81-2.54 (m, 6H) and 1.13 (t, 6H, J=6.0); ESMS m/e: 512 (M+H)⁺.

The following compounds were prepared analogously:

### EXAMPLE 6b

### 1-(3-{3-[(2S)-2-(Methoxymethyl)pyrrolidinyl]-2-hydroxypropoxy}phenyl)-3-{[3-(trifluoromethyl)phenyl]azamethylene}benzo[d]azolidin-2-one

ESMS m/e: 554 (M+H)⁺.

### EXAMPLE 6c

### 1-(3-{2-Hydroxy-3-[(2-methoxyethyl)methylamino]propoxy}phenyl)-3-{[3-(trifluoromethyl)phenyl]azamethylene}benzo[d]azolidin-2-one

ESMS m/e: 528 (M+H)⁺.

### EXAMPLE 6d

### 1-[2-(3-{[2-(Dimethylamino)ethyl]methylamino}-2-hydroxypropoxy)phenyl]-3-{[3-(trifluoromethyl)phenyl]azamethylene}benzo[d]azolidin-2-one

ESMS m/e: 541 (M+H)⁺.

### EXAMPLE 6e

### 1-{3-[(2R)-3-(Diethylamino)-2-hydroxypropoxy]phenyl}-3-{[3-(trifluoromethyl)phenyl]azamethylene}benzo[d]azolidin-2-one

ESMS m/e: 512 (M+H)⁺.

### EXAMPLE 6f

### 1-{3-[(2S)-3-(Diethylamino)-2-hydroxypropoxy]phenyl}-3-{[3-(trifluoromethyl)phenyl]azamethylene}benzo[d]azolidin-2-one

ESMS m/e: 512 (M+H)⁺.

Formulations

The pharmaceutical formulations of the invention may be prepared by conventional methods in the art.

For example, tablets may be prepared by mixing the active ingredient with ordinary adjuvants and/or diluents and subsequently compressing the mixture in a conventional tabletting machine may prepare tablets. Examples of adjuvants or diluents comprise: corn starch, potato starch, talcum, magnesium stearate, gelatine, lactose, gums, and the like. Any other adjuvants or additives usually used for such purposes such as colorings, flavorings, preservatives etc. may be used provided that they are compatible with the active ingredients.

1) Tablets Containing 5.0 mg of Compound 3d Calculated as the Free Base:

| | | |
|---|---|---|
| Compound 3d | 5.0 | mg |
| Lactose | 60 | mg |
| Maize starch | 30 | mg |
| Hydroxypropylcellulose | 2.4 | mg |
| Microcrystalline cellulose | 19.2 | mg |
| Croscarmellose Sodium Type A | 2.4 | mg |
| Magnesium stearate | 0.84 | mg |

2) Tablets Containing 0.5 mg of Compound 3d Calculated as the Free Base:

| | |
|---|---|
| Compound 3d | 0.5 mg |
| Lactose | 46.9 mg |
| Maize starch | 23.5 mg |
| Povidone | 1.8 mg |
| Microcrystalline cellulose | 14.4 mg |
| Croscarmellose Sodium Type A | 1.8 mg |
| Magnesium stearate | 0.63 mg |

3) Syrup Containing 25 mg of Compound 3d per Milliliter:

| | | |
|---|---|---|
| Compound 3d | 25 | mg |
| Sorbitol | 500 | mg |
| Hydroxypropylcellulose | 15 | mg |
| Glycerol | 50 | mg |
| Methyl-paraben | 1 | mg |
| Propyl-paraben | 0.1 | mg |
| Ethanol | 0.005 | mL |
| Flavor | 0.05 | mg |
| Saccharin | 0.5 | mg |
| Water | 1 | mL |

In Vitro Methods

The pharmacological properties of the compounds of the present invention were evaluated at the cloned human GAL₃ receptor using the protocols disclosed in U.S. Pat. No. 6,329,197, the contents of which are hereby incorporated by reference.

Using this protocol, the binding by the compound to a radiolabeled ligand (¹²⁵I-labeled porcine galanin) to membranes of human cloned GAL₃ receptors expressed in CHO cells was determined in vitro.

Briefly, the affinity of the compounds was measured by their ability to displace ¹²⁵I-labeled porcine galanin by incubating GAL₃ receptor expressing membranes with the compound and radioligand at 30° C. for 1 h. The binding affinities of the compounds may be determined in equilibrium competition assays, using 0.1-0.5 nM radioligand in the presence of e.g., twelve different concentrations of the displacing ligands. Incubation was terminated by rapid vacuum filtration over GF/B filters treated with 0.5% polyethyleneimine using a cell harvester.

The binding affinities for the compounds in the present invention, exemplified above, at the GAL₃ receptor were determined to be 200 nM or less. For the majority of the compounds, the Ki values are 100 nM or less, and for a large group of compounds the Ki values are 25 nM or less.

What is claimed:

1. A compound having the structure: 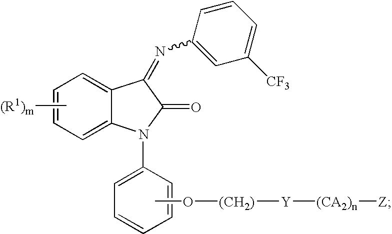
wherein each R¹ is independently straight chained or branched C₁-C₄ alkyl, straight chained or branched C₁-C₄ alkoxy, CN, F, Cl, Br or I;
wherein each A is independently H, hydroxyl or straight chained or branched C₁-C₄ alkyl;
wherein Y is —CH=CH—, —(CA₂)ₜ(NR⁵)(CH₂)— or null;
wherein Z is —N(R²)(R³), 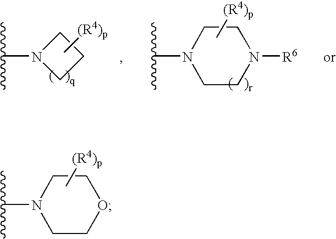
wherein R² is H, straight chained or branched C₁-C₄ dialkyl ether or straight chained or branched C₁-C₇ alkyl, wherein the C₁-C₇ alkyl may be substituted with CN or hydroxyl;
wherein R³ is H, straight chained or branched C₁-C₄ dialkyl ether or straight chained or branched C₁-C₇ alkyl, wherein the C₁-C₇ alkyl may be substituted with CN or hydroxyl or 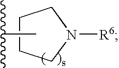
wherein each R⁴ is independently straight chained or branched C₁-C₄ dialkyl ether or straight chained or branched C₁-C₄ alkyl, wherein the C₁-C₄ alkyl may be substituted with CN or hydroxyl;
wherein R⁵ is H or straight chained or branched C₁-C₄ alkyl;
wherein R⁶ is H or straight chained or branched C₁-C₄ alkyl;
wherein m is an integer from 0 to 4 inclusive;
wherein n is an integer from 1 to 5 inclusive;
wherein p is an integer from 0 to 4 inclusive;
wherein q is an integer from 0 to 3 inclusive;
wherein r is 1 or 2;
wherein s is 1 or 2; and
wherein t is an integer from 1 to 3 inclusive;
or a pharmaceutically acceptable salt thereof.

2. The compound of claim 1, wherein each R¹ is independently methyl, ethyl, propyl, CN, F, Cl or Br.

3. The compound of claim 2, wherein each A is independently H, hydroxyl, methyl or ethyl.

4. The compound of claim 3, wherein n is an integer from 1 to 3 inclusive.

5. The compound of claim 4, wherein Z is —N(R²)(R³).

6. The compound of claim 5, wherein R² is straight chained C₁-C₄ dialkyl ether or straight chained C₁-C₄ alkyl, wherein the C₁-C₄ alkyl may be substituted with CN or hydroxyl; and
wherein R³ is straight chained C₁-C₄ dialkyl ether or straight chained C₁-C₄ alkyl, wherein the C₁-C₄ alkyl may be substituted with CN or hydroxyl.

7. The compound of claim 6, wherein Y is null or —CH═CH—.

8. The compound of claim 7, wherein Y is null.

9. The compound of claim 4, wherein Z is

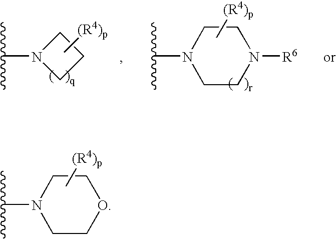

10. The compound of claim 9, wherein each R⁴ is independently straight chained C₁-C₄ dialkyl ether or straight chained C₁-C₄ alkyl.

11. The compound of claim 10, wherein Y is null or —CH═CH—.

12. The compound of claim 11, wherein Z is

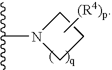

13. The compound of claim 12, wherein Y is null and q is 2.

14. The compound of claim 11, wherein Z is

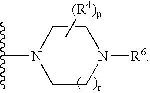

15. The compound of claim 14, wherein Y is null and R⁶ is H or methyl.

16. The compound of claim 11, wherein Z is

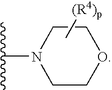

17. The compound of claim 16, wherein Y is null and p is 0.

18. A pharmaceutical composition comprising a therapeutically effective amount of a compound of claim 1 and a pharmaceutically acceptable carrier.

19. A process for making a pharmaceutical composition comprising admixing a therapeutically effective amount of a compound of claim 1 and a pharmaceutically acceptable carrier.

20. A method of treating a subject suffering from depression comprising administering to the subject a therapeutically effective amount of the compound of claim 1.

21. A method of treating a subject suffering from anxiety comprising administering to the subject a therapeutically effective amount of the compound of claim 1.
